(19) United States
(12) Patent Application Publication
(10) Pub. No.: US 2004/0122377 A1
(43) Pub. Date: Jun. 24, 2004

(54) Syringe delivery tip adapted to provide controlled flow rate

(76) Inventors: Dan E. Fischer, Sandy, UT (US); Paul E. Lewis, Midvale, UT (US)

Correspondence Address: Rick D. Nydegger, WORKMAN, NYDEGGER & SEELEY, 1000 Eagle Gate Tower, 60 East South Temple, Salt Lake City, UT, 84111, US

(21) Appl. No.: 10/324,249

(22) Filed: Dec. 19, 2002

(51) Int. Cl. A61M 5/00; A61C 5/04

(52) U.S. Cl. 604/239; 433/89

(57) ABSTRACT

A delivery tip configured to engage a syringe delivery device includes a hub, a cannula attached to the hub, and structure configured to regulate the fluid flow rate through the delivery tip. The hub includes a passage disposed there through. The cannula includes a lumen formed therethrough in communication with the hub passage. A restriction portion is formed in the hub passage. The restriction portion is such that it forms a minimum diameter in the hub passage that is smaller than the minimum diameter of the lumen. As such, the fluid rate is primarily determined by the size of the restriction portion rather than the diameter of the cannula lumen.

### BACKGROUND OF THE INVENTION

1. The Field of the Invention

The present invention relates to syringe delivery tips adapted for use with a syringe. More particularly, the present invention relates to syringe delivery tips that provide for controlled flow rate when extruding flowable compositions from a syringe.

2. The Relevant Technology

Syringe devices are well known in the medical field. Particularly, in the dental industry, syringe devices are commonly used to deliver various dental compositions. It is desirable, in some cases, for dental compositions to be applied at a controlled flow rate. A controlled flow rate allows for reproducible results so that a dentist or dental technician can predict with more certainty the amount of pressure on a syringe plunger that is needed to result in the expulsion of a desired amount of dental composition. Previous delivery tips have not provided a controlled flow rate so that even when the same amount of pressure is applied to the same delivery tip, a varied amount of dental composition is extruded. Furthermore, different-sized cannulas on different delivery tips result in different flow rates, even though the dentist or dental technician may desire a similar flow rate for differently sized cannulas.

In addition, in dental procedures, the worksite of the patients' mouth tends to be remote and difficult to reach because of space limitations. In many dental procedures, it is important to deliver small, discrete quantities of fluid to these sites. As previously mentioned, it is often difficult to control the delivery of the fluid to the specific site.

FIG. 1A shows one prior art delivery tip configured to restrict flow rate. FIG. 1A illustrates a delivery tip 10 which is configured to engage the discharging end of a syringe. Delivery tip 10 may be selectively or permanently coupled to the syringe. Delivery tip 10 has a proximal end 12 and a distal end 14. A hub 16 is formed at the proximal end 12 of delivery tip 10. A cannula 18 is attached to hub 16 and terminates at distal end 14 of delivery tip 10. Cannula 18 has a bundle of fibers 20 disposed at its distal end 14. In order to retain the fibers 20 within the distal end 14 of the cannula 18, the cannula 18 is typically crimped near its distal end 14. The crimp consists of one or more indentations which slightly deform the distal end of cannula 18 so that fibers 20 are securely disposed therein.

With reference to FIG. 1B, hub 16 comprises a passage 22 which, when delivery tip 10 is coupled to a syringe, is in fluid communication with the interior of the syringe. Cannula 18 has a lumen 24 formed therein. Passage 22 is in fluid communication with lumen 24 of cannula 18. Passage 22 generally decreases in diameter from the proximal end to the distal end of hub 16 until passage 22 connects to lumen 24 of cannula 18.

The fibers 20 disposed in lumen 24 of cannula 18 assist in applying the composition and provide the additional feature of restricting fluid flow by blocking the distal end of cannula 18. Whereas the flow restriction provided by the fibers 20 may be beneficial in some cases, variations in the number, size, and distribution of fibers within the distal end 14 of the cannula 18 results in varying flow rate from tip to tip. Changes in the inner cannula diameter can also greatly affect flow rate, as can differences in the crimp among cannulas of the same size. Such variations can lead to unpredictable results, such as too much or too little dental composition being discharged onto the targeted site. Thus, variations in the flow rate can impede the ability of the dentist or technician to perform a particular dental procedure in the manner desired.

In view of the foregoing, there is currently a need in the art for improved syringe delivery systems and, more particularly, to syringe delivery tips that are configured to provide a more controlled flow rate such that the user is provided with increased control over the delivery of a composition.

### BRIEF SUMMARY OF THE INVENTION

The present invention relates generally to syringes having delivery tips configured to control the fluid flow rate therethrough. Specifically, the present invention is directed to controlling the fluid flow rate of a flowable composition being extruded through the distal end of a delivery tip by providing a restriction portion in the hub of the delivery tip. The delivery tips of the present invention are configured to either be selectively coupled or permanently attached to a syringe delivery system.

The delivery tips of the present invention generally comprise a proximal end and a distal end, the proximal end forming a hub and the distal end terminating in a cannula. A passage is formed in the hub and a lumen is formed in the cannula, the passage being in fluid communication with the lumen of the cannula. The passage in the hub includes a restriction portion that is designed to limit and control the fluid flow rate through the delivery tip. The restriction portion is any structure which causes the minimum diameter of the passage to be smaller than the minimum diameter of the lumen of the cannula. Thus, the fluid flow rate is determined primarily by the size of the restriction portion rather than the size of the lumen through the cannula. The restriction portion may be disposed anywhere in the hub.

It is contemplated that the restriction mechanism of the present invention may be used in conjunction with any cannula. For example, the cannulas of the present invention may be a metal cannula, a needle, a flocked cannula, cannula having fibers disposed therein, and other cannulas known in the art.

These and other advantages and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1A illustrates a prior art delivery tip having a hub and a cannula with a bundle of fibers disposed in an end thereof;

FIG. 1B illustrates a cross-sectional view of the delivery tip of FIG. 1A;

FIG. 2 illustrates an exploded view of an exemplary syringe delivery system configured to engage an embodiment of a delivery tip of the present invention;

FIG. 3A illustrates a cross-sectional view of one embodiment of a delivery tip according to the invention;

FIG. 3B illustrates a cross-sectional view of another embodiment of a delivery tip according to the invention;

FIG. 3C illustrates a cross-sectional view of an embodiment of a delivery tip of the present invention in which the delivery tip is formed integrally with the barrel of a syringe;

FIG. 4A illustrates a delivery tip according to the invention comprising a needle;

FIG. 4B illustrates a delivery tip according to the invention comprising a cannula with fibers disposed through an end thereof; and

FIG. 4C illustrates a delivery tip according to the invention having a cannula with a fiber flocked end.

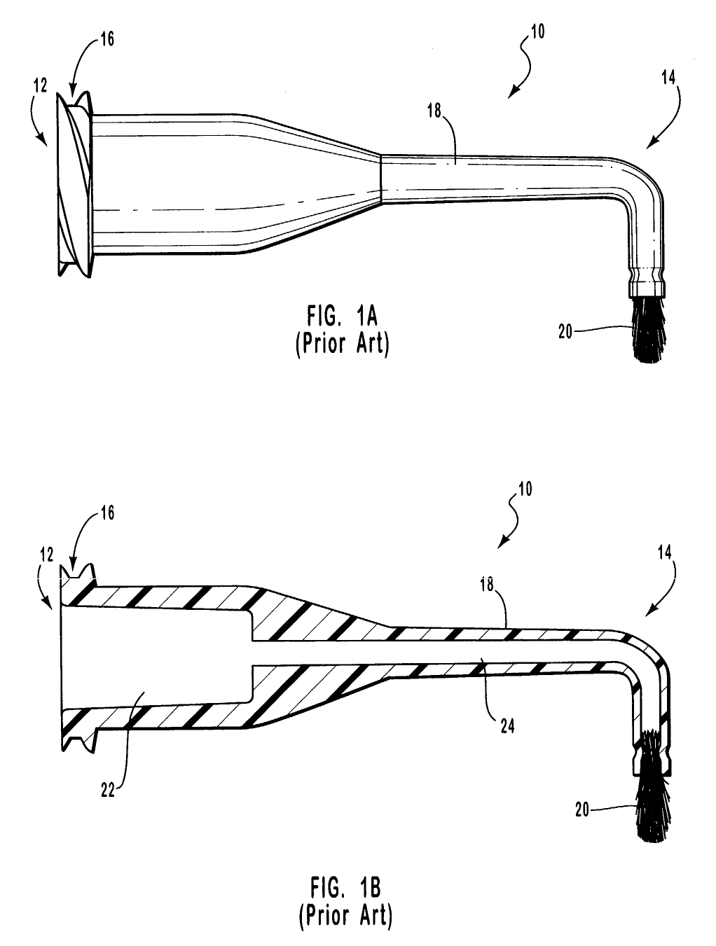

### DETAILED DESCRIPTION OF THE PREFERRED EMBODIMENTS

The present invention relates to delivery tips for syringe delivery systems configured to control the flow of fluid material therethrough. In each embodiment of the invention, the delivery tips extend from, or are configured to engage the outlet of, a syringe. The delivery tips of the present invention may be either integrally connected to or selectively couplable to a syringe. In order to provide context for interpreting the scope of the invention, certain terms will now be defined.

The term “composition,” as used herein, refers to any fluid material or fluid composition of materials capable of being dispensed through a syringe. By way of example and not limitation, the compositions referred to herein include organic and synthetic compositions as well as water and solvent based compositions. Although the terms “composition” and “fluid material” are used interchangeably herein, it will be appreciated that the compositions and fluid materials are not limited to having any particular viscosity. Rather the viscosity of the fluid materials can vary to accommodate different needs and preferences.

The term “hub,” as defined herein, refers to any connector portion to which a cannula is directly attached. A hub may or may not be formed integrally witn a syringe. When formed integrally as part of a syringe, the hub is defined as the portion of the syringe to which a cannula is attached and the portion of the syringe within which the plunger can be selectively advanced or withdrawn.

The term “cannula,” as defined herein, refers to any tip, tube, needle, and other dispensing device configured to dispense a fluid material and is characterized by the attribute of including at least a hollow or concave portion through which the fluid material can flow. The cannula is also characterized by being attached to a syringe by a hub.

The term “mating engagement formations,” as defined herein, refers to any combination of engaging formations, including, but not limited to, recesses, ridges, protrusions, holes, latches, clips, knobs, pins, slots, tabs, and apertures which are configured to interconnect, interest, mate, lock, or otherwise frictionally engage.

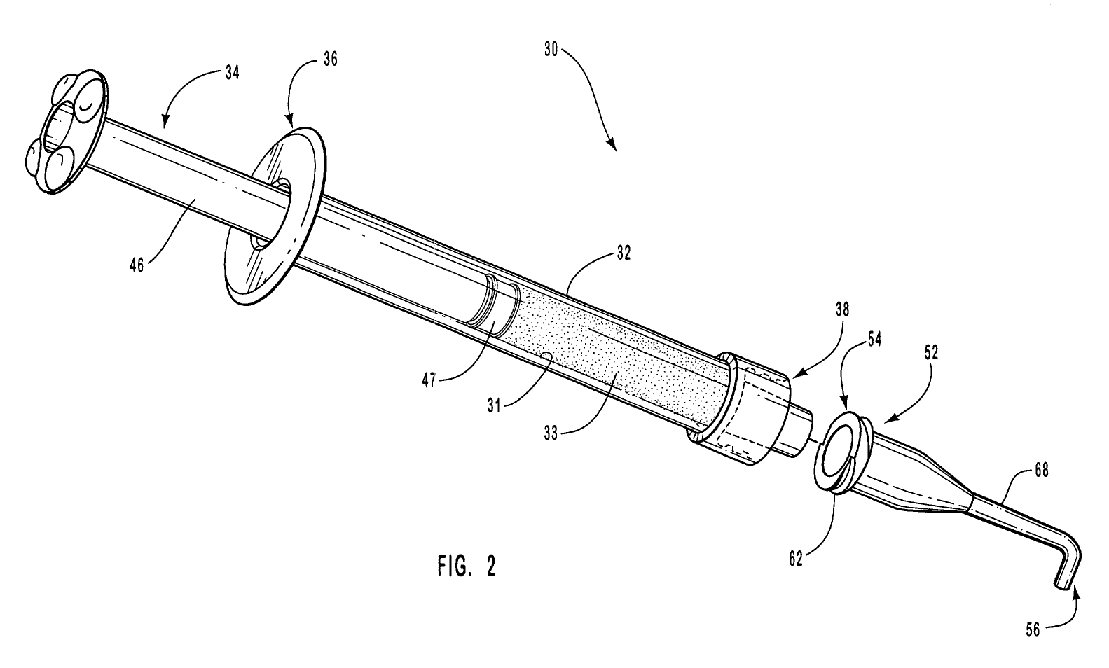

With reference now to FIG. 2, an exemplary syringe delivery system is shown being engageable with a delivery tip incorporating features of the present invention. It will be appreciated that the delivery tips of the present invention may be employed with any syringe delivery device known in the art. Thus, the following is provided by way of example and not by limitation.

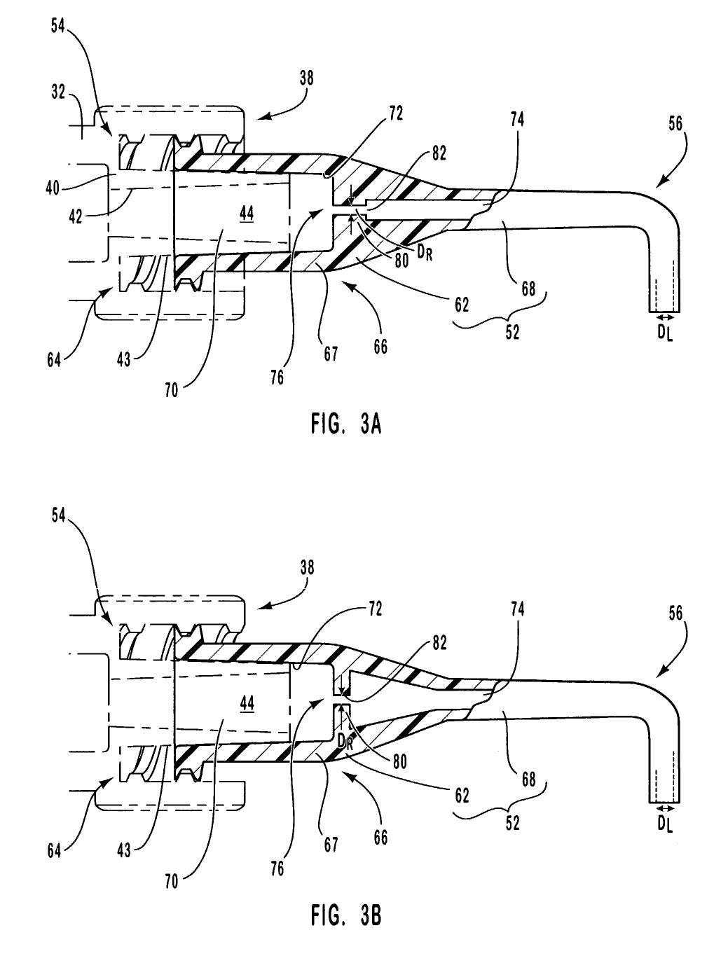

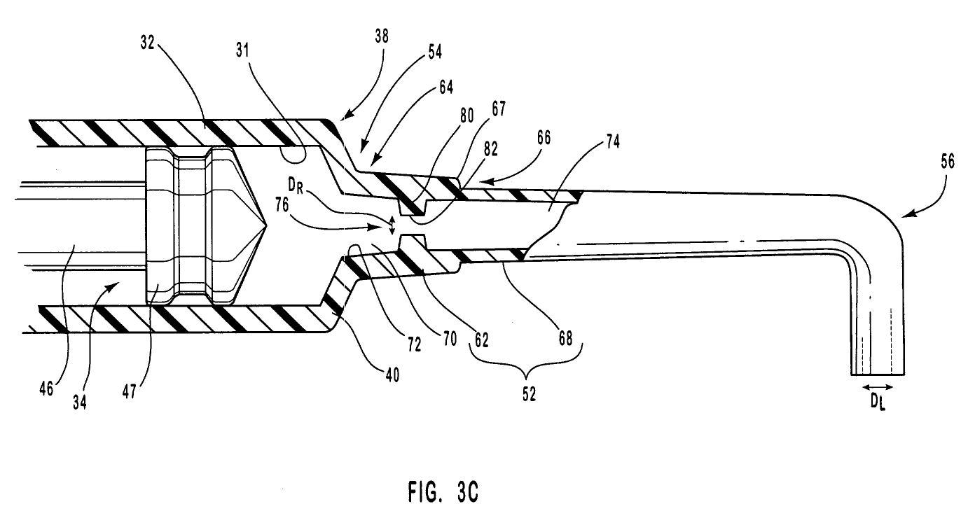

As shown in FIG. 2, a syringe 30 is engageable with a delivery tip 52. Syringe 30 comprises a barrel 32 configured to receive a plunger 34 therein, both being sized and configured so as to cooperate together. Barrel 32 comprises an elongated tubular body with a proximal end 36 and distal end 38. Barrel 32 has an interior surface 31 which defines a fluid reservoir within barrel 32. Barrel 32 is thus configured to store and deliver a composition 33 disposed therein. Plunger 34 comprises an elongated shaft 46 and resilient sealing plug 47 (FIGS. 2 and 3C).

FIG. 2 illustrates that distal end 38 of barrel 32 is configured to engage a delivery tip 52. As seen in FIG. 3A, at distal end 38 of barrel 32, an annular shoulder 40 extends transversely around barrel 32. An elongated and hollow neck 42 extends substantially perpendicularly from annular shoulder 40. The inside diameter of neck 42 defines a conduit 44. Conduit 44 is thus in fluid communication with barrel 32.

Delivery tip 52 will now be described in further detail. As shown in FIG. 2, delivery tip 52 has a proximal end 54 and a distal end 56. Proximal end 54 of delivery tip 52 is capable of being connected to distal end 38 of barrel 32. As shown best in FIGS. 3A and 3B, neck 42 of barrel 32 may be configured to have a luer taper 43 that engages with a corresponding luer taper 72 formed on proximal end 54 of delivery tip 52. Luer tapers 43, 72 on barrel 32 and delivery tip 52, respectively, are one example of structure capable of performing the function of a coupling means for attaching delivery tip 52 to barrel 32. Various other embodiments of a coupling means are equally effective in carrying out the intended function thereof. By way of example and not limitation, any of the mating engagement formations defined above may be employed as coupling means.

In the alternative embodiment of FIG. 3C, delivery tip 52 may instead be integrally attached to distal end 38 of barrel 32 by injection molding, an adhesive or other integral connection. Where delivery tip 52 is formed integrally with barrel 32, proximal end 54 of delivery tip 52 merges with the distal end 389 of the syringe barrel 32. As illustrated in the embodiment shown in FIG. 3C, proximal end 54 of delivery tip 52 terminates at annular shoulder 40.

Returning to FIG. 3A, delivery tip 52 is shown in a detailed cross-sectional view. Delivery tip 52 comprises a hub 62 having a cannula 68 attached thereto. Hub 62 has a proximal end 64 and a distal end 66. Distal end 66 of hub 62 is configured to engage with cannula 68. As discussed above, hub 62 is configured to engage distal end 38 of barrel 32. As shown in FIG. 2, hub 62 is substantially cylindrical in configuration; however, other cross-sectional configurations may be equally applicable. At distal end 66 of hub 62, an annular shoulder 67 extends transversely around hub 62. Cannula 68 engages annular shoulder 67 of hub 62. In one embodiment, hub 62 is selectively couplable to cannula 68 such that cannula 68 may be interchanged with other cannulas, as will be described later in further detail. Alternatively, hub 62 may be integrally connected to cannula 68. Hub 62 may be attached to cannula 68 in a variety of ways including, but not limited to, any of the mating engagement formations defined above.

Hub 62 comprises a passage 70 disposed therethrough. Passage 70 has an inner surface 72, a portion of which may be configured to sealingly mate with a corresponding surface 43 on neck 42 of barrel 32 in those embodiments where delivery tip 52 is selectively coupled thereto. In the embodiments where passage 70 forms part of a luer connection, passage 70 has a substantially tapered surface 72; however, other cross-sectional configurations may be applicable depending on various needs or preferences in the design of syringe 30. The degree to which passage 70 tapers and whether the passage 70 actually tapers will depend in large extent on the configuration of the corresponding coupling structure on barrel 32. When hub 62 is engaged with barrel 32, passage 70 is in fluid connection with conduit 44 such that composition 33 from barrel 32 (FIG. 2) is able to flow therethrough.

As seen in FIGS. 3A and 3B, cannula 68 comprises a lumen 74 formed therethrough. Lumen 74 is in fluid communication with passage 70 of hub 62 to allow composition 33 to flow through cannula 68. Lumen 74 is shown as having a substantially circular cross-section; however other cross-sectional configurations are possible and may be selected depending on various needs or preferences in the design of delivery tip 52. In addition, in some embodiments, lumen 74 may taper from a proximal end to a distal end of cannula 68. Lumen 74 is shown as having a minimum diameter Dₗ, which, in the embodiment of FIG. 3A, is taken to be the distal-most end of lumen 74. However, it will be appreciated that the minimum diameter Dₗ of lumen 74 may occur anywhere along cannula 68.

With reference to FIGS. 3A through 3C, a restriction portion 76 is formed in passage 70 of hub 62. Restriction portion 76 is any structure that causes passage 70 to have a minimum diameter Dᵣ that is smaller than the minimum diameter Dₗ of lumen 74 of cannula 68. In one embodiment, restriction portion 76 comprises a wall 80 formed substantially transversely relative to passage 70 of hub 62. Wall 80 comprises an aperture 82 formed therein having a diameter Dᵣ that remains substantially constant i.e., it does not change in response to changes in the pressure of fluids passing therethrough. Restriction portion 76 may be formed with hub 62 by mechanical connection or injection molding.

It will be appreciated that restriction portion 76 may be formed anywhere along passage 70 of hub 62. In the embodiment of FIG. 3A, wall 80 is formed at distal end 66 of hub 62. Alternatively, in the embodiment of FIG. 3B, wall 80 is formed between proximal end 64 and distal end 66 of hub 62. In this embodiment, wall 80 bifurcates passage 70, with passage 70 extending from both sides of wail 80. In another embodiment (not shown), restriction portion 76 may be formed through the entire passage 70 of hub 62, such that the minimum diameter Dr of restriction portion 76 is the same throughout.

Significantly, the minimum diameter Dᵣ of restriction portion 76 is smaller than the minimum diameter Dₗ of lumen 74 of cannula 68. In one embodiment Dᵣ is 75% or less of Dₗ. In another embodiment, Dᵣ is 50% or less of Dₗ. In still another embodiment, Dᵣ is 25% or less of Dₗ. In one embodiment, aperture 82 has a diameter Dᵣ of about 0.1 mm to about 2 mm. Because Dᵣ is always smaller than Dₗ, Dᵣ will be the primary controlling factor governing the rate of fluid flow through cannula 68.

From the foregoing, it will be appreciated that restriction portion 76 provides means for maintaining a constant fluid flow through lumen 74 of cannula 68 regardless of the diameter of the lumen 74 of cannula 68. While the pressure applied to plunger 34 may also control the rate of fluid flow, restriction portion 76 provides the primary mechanism for maintaining a constant fluid flow rate exiting lumen 74 of cannula 68. Thus, as fluid flows through barrel 32, through conduit 44 and into passage 70 of hub 62, restriction portion 76 is the primary or sole restriction mechanism in fluid communication with barrel 32.

While some embodiments of cannula 68 may comprise a configuration designed to restrict fluid flow (such as fibers embedded inside lumen 74), it will be appreciated that the fluid flow is primarily controlled by restriction portion 76 in hub 62 before reaching the distal end of cannula 68. That is, restriction portion 76 primarily defines the flow rate exiting cannula 68.

As discussed above, hub 62 is configured to engage cannula 68. Cannula 68 may be formed integrally with hub 62 or may be selectively couplable or selectively interchangeable with hub 62. Where hub 62 is selectively couplable to cannula 68, it will be appreciated that hub 62 already comprises a restriction portion 76 such that a constant flow rate will be provided regardless of the diameter of cannula 68 that is selectively coupled to hub 62.

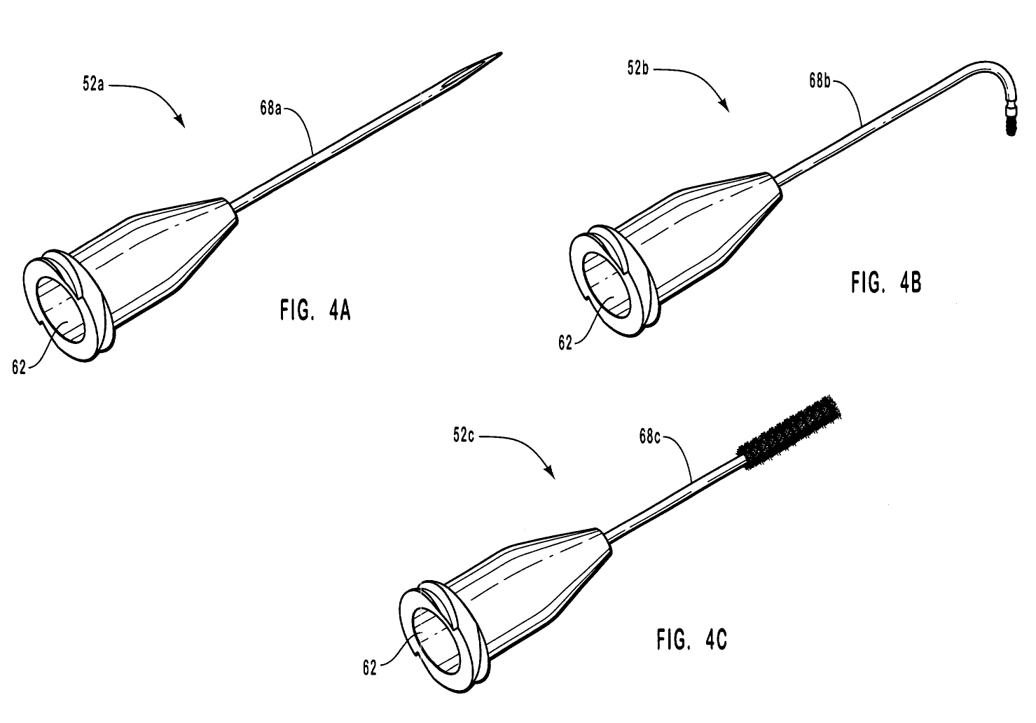

Turning now to FIGS. 4A through 4C, various embodiments of cannulas that may be used in the delivery tips of the present invention are shown. It will be appreciated that cannulas 52a through 52c in FIGS. 4A through 4C are merely exemplary of cannulas which may be used in the present invention, and not be understood as limiting in any way. FIG. 4A depicts delivery tip 52a having cannula 68a configured as a needle. FIG. 4B illustrates delivery tip 52b having a curved or bent tip cannula 68a having a plurality of fibers disposed in a distal end thereof. FIG. 4C illustrates delivery tip 52c where cannula 68c has flocked fibers on an end thereof. Significantly, FIGS. 4A through 4C illustrates that different types of cannulas may have the same hub 62 configured to control fluid flow rate therethrough. Thus, in one embodiment, cannulas 68a through 68c are interchangeable with the same hub 62 having restriction portion 76 during manufacture. In this embodiment, it will be appreciated that the same fluid flow rate will be exhibited in each delivery tip 52a through 52c, despite the different types and/or sizes of cannulas employed thereon, as long as the restriction 76 is the same.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A delivery tip adapted for use with a syringe delivery device, the delivery tip comprising:
a hub configured so as to engage an end of a syringe delivery device, the hub having a proximal end, a distal end, and a passage extending therethrough;
a cannula connected to the distal end of the hub and having a lumen in fluid communication with the hub passage; and
a restriction portion within the hub passage having a diameter that is smaller than the diameter of the cannula lumen.

2. The delivery tip as recited in claim 1, the delivery tip being configured so as to be selectively couplable to the syringe delivery device.

3. The delivery tip as recited in claim 1, the delivery tip being integrally connected to the syringe delivery device.

4. The delivery tip as recited in claim 1, the diameter of the restriction portion being about 75% or less than the diameter of the cannula lumen.

5. The delivery tip as recited in claim 1, the diameter of the restriction portion being about 50% or less than the diameter of the cannula lumen.

6. The delivery tip as recited in claim 1, the diameter of the restriction portion being about 25% or less than the diameter of the cannula lumen.

7. The delivery tip as recited in claim 1, the restriction portion comprising a wall formed substantially transversely to the hub passage, the wall having an aperture therethrough that forms a minimum diameter through the hub passage.

8. The delivery tip as recited in claim 1, the diameter of the restriction portion remaining constant regardless of changes in fluid pressure when a fluid passes therethrough.

9. The delivery tip as recited in claim 1, the cannula comprising a lumen having a constant diameter.

10. The delivery tip as recited in claim 1, the cannula comprising a lumen having varying diameter, including a minimum diameter that is greater than the diameter of the restriction portion.

11. The delivery tip as recited in claim 1, further comprising a plurality of fibers extending through a portion of the cannula lumen.

12. The delivery tip as recited in claim 1, further comprising a plurality of fibers attached to an outer surface of the cannula

13. A delivery tip adapted to be selectively coupled with a syringe delivery device, the delivery tip comprising:
a hub that includes coupling means for selectively coupling the hub to a syringe delivery device, the hub having a proximal end, a distal end, and a passage extending therethrough;
a cannula connected to the distal end of the hub and having a lumen in fluid communication with the hub passage; and
a restriction portion within the hub passage having a diameter that is smaller than the diameter of the cannula lumen.

14. The delivery tip as recited in claim 13, the coupling means comprising a luer taper within at least a portion of the hub passage.

15. The delivery tip as recited in claim 13, the coupling means comprising coupling threads disposed on an outer surface of the hub.

16. The delivery tip as recited in claim 13, the restriction portion comprising a wall formed substantially transversely to the hub passage, the wall having an aperture therethrough that forms a minimum diameter through the hub passage.

17. A syringe delivery device configured to store and deliver a fluid, the syringe delivery device comprising:
a syringe barrel having a proximal end, a distal end, and an interior surface defining a fluid reservoir therein; and
a delivery tip integrally attached to the proximal end of the syringe barrel; the delivery tip comprising:
a hub having a proximal end that is integrally attached to the proximal end of the syringe barrel, a distal end, and a passage extending therethrough;
a cannula connected to the distal end of the hub and having a lumen in fluid communication with the hub passage; and
a restriction portion within the hub passage having a diameter that is smaller than the diameter of the cannula lumen.

18. The syringe delivery device as recited in claim 17, the diameter of the restriction portion remaining constant regardless of changes in fluid pressure when a fluid passes the therethrough.

19. A syringe delivery system for storing and delivering a fluid, the syringe delivery system comprising:
a syringe barrel having a proximal end, a distal end, and an interior surface defining a fluid reservoir therein; and
a delivery tip selectively couplable to the proximal end of the syringe barrel; the delivery tip comprising:
a hub that includes coupling means for selectively coupling the hub to the proximal end of the syringe barrel, the hub having a proximal end, a distal end, and a passage extending therethrough;
a cannula connected to the distal end of the hub and having a lumen in fluid communication with the hub passage; and
a restriction portion within the hub passage having a diameter that is smaller than the diameter of the cannula lumen.

20. The syringe delivery system as recited in claim 19, the coupling means comprising at least one of a luer taper within at least a portion of the hub passage or coupling threads disposed on an outer surface of the hub.
